(19) United States
(12) Patent Application Publication
(10) Pub. No.: US 2010/0292778 A1
(43) Pub. Date: Nov. 18, 2010

(54) EXPANDABLE STENT COMPRISING END MEMBERS HAVING AN INTERLOCKING CONFIGURATION

(75) Inventors: Blayne A. Roeder, Lafayette, IN (US); Seoggwan Kim, West Lafayette, IN (US); Richard A. Swift, South Bend, IN (US)

Correspondence Address: BRINKS HOFER GILSON & LIONE/CHICAGO/COOK, PO BOX 10395, CHICAGO, IL, 60610, US

(73) Assignee: MED Institute, Inc., West Lafayette, IN (US)

(21) Appl. No.: 12/467,047

(22) Filed: May 15, 2009

(51) Int. Cl. A61F 2/06

(52) U.S. Cl. 623/1.17

(57) ABSTRACT

An expandable stent for use in a body vessel comprises a thin-walled tubular framework including two or more circumferentially adjacent end members extending in a longitudinal direction from an end of the framework. In a delivery configuration of the tubular framework, the end members have an interlocking configuration. Each end member has a first interlocking side and a second interlocking side, where the first interlocking side has a circumferentially directed protrusion and the second interlocking side has a circumferentially directed recess. The protrusion of a first end member mates with the recess of a second end member. In an expanded configuration of the tubular framework, the end members are disengaged from the interlocking configuration. Each end member may be an eyelet including an opening for a radiopaque rivet. A method of preparing the expandable stent for delivery into a body vessel is also described.

### TECHNICAL FIELD

The present disclosure is related generally to expandable stents and more particularly to expandable stents including radiopaque markers.

### BACKGROUND

Stents are tubular support structures that can be used in a variety of medical procedures to treat blockages, occlusions, narrowing ailments and other problems that restrict flow through body vessels. Expandable stents are radially compressed for delivery into a body vessel and then radially expanded once in place at a treatment site, where the tubular support structure of the stent contacts and supports the inner wall of the vessel. Such stents may expand in response to the inflation of a balloon (balloon-expandable stents), or they may expand spontaneously when released from a delivery device (self-expanding stents). Metal alloys such as stainless steel and Nitinol are commonly used to form expandable stents.

Numerous vessels throughout the vascular system, including peripheral arteries, such as the carotid, brachial, renal, iliac and femoral arteries, and other vessels, may benefit from treatment by a stent. In order to effectively treat diseased vessels, it is important that the stent be precisely placed at the site of the constriction. One approach to achieving precise stent placement is to improve the “visibility” or imageability of the stent under x-ray irradiation. Radiopaque markers made at least in part of heavy metals such as platinum or gold, which produce higher x-ray contrast than stainless steel or Nitinol, may thus be attached to ends of the stent. FIG. 1 shows an exemplary stent 100 including body cells 115, end cells 120, and eyelets 125 extending from the end cells 120. The eyelets 125 are sized to hold radiopaque rivets made of an x-ray opaque material. Radiopaque markers are thus formed from the eyelet-rivet structure. During the implantation procedure, the position of the radiopaque markers—and thus the location of the stent 100 in the body vessel—may be readily monitored using an x-ray fluoroscope.

In preparation for delivery into a body vessel, an expandable stent is radially compressed into a low-profile (reduced diameter) configuration. Typically, the compression is carried out in a crimping device or other apparatus configured to apply a radial force to an outer surface of the stent. A push rod may be used to advance the compressed stent out of the crimping device and into a transfer tube or sheath for transport into a vessel. In the case of expandable stents including radiopaque markers at the ends, such as the stent 100 shown in FIG. 1, the push rod contacts the eyelets 125 containing the radiopaque rivets as the stent 100 is being advanced through the compression apparatus. The axial force applied to the eyelets 125 may cause the eyelets 125 and/or the end cells 120 to bend or twist, thus distorting the shape of the compressed stent 100.

### BRIEF SUMMARY

An expandable stent having an improved design and a method of preparing the stent for delivery into a body vessel are described herein. The expandable stent can be transferred from a compression apparatus to a transfer tube or other tubular delivery system without buckling of end members (e.g., eyelets for containing radiopaque rivets) of the stent.

The expandable stent comprises a thin-walled tubular framework including two or more circumferentially adjacent end members extending in a longitudinal direction from an end of the framework. In a delivery configuration of the tubular framework, the end members have an interlocking configuration. Each end member has a first interlocking side and a second interlocking side, where the first interlocking side has a circumferentially directed protrusion and the second interlocking side has a circumferentially directed recess. The protrusion of a first end member mates with the recess of a second end member. In an expanded configuration of the tubular framework, the end members are disengaged from the interlocking configuration.

The method of preparing an expandable stent for delivery into a body vessel comprises inserting an expandable stent into a compression apparatus, where the expandable stent comprises a thin-walled tubular framework including two or more circumferentially adjacent end members extending in a longitudinal direction from an end of the framework. Each end member has a first interlocking side and a second interlocking side, where the first interlocking side has a circumferentially directed protrusion and the second interlocking side has a circumferentially directed recess. The stent is compressed from the first diameter to a second diameter smaller than the first diameter. A circumferentially continuous edge is created at an end of the stent, where the circumferentially continuous edge includes an edge of each end member. An axially directed force is applied to the circumferentially continuous edge to advance the stent through the compression apparatus for removal therefrom.

### BRIEF DESCRIPTION OF THE DRAWINGS

FIG. 1 shows an exemplary prior art stent including eyelets extending from end cells thereof;

FIG. 2A shows a perspective view of a portion of an expandable stent including interlocking eyelets according to a first embodiment;

FIG. 2B shows a flattened plan view of the portion of the expandable stent of FIG. 2A;

FIG. 2C shows an end view of the expandable stent of FIG. 2A in a fully compressed configuration;

FIG. 3A shows a perspective view of a portion of an expandable stent including interlocking eyelets according to a second embodiment;

FIG. 3B shows a flattened plan view of the portion of the expandable stent of FIG. 3A;

FIG. 3C shows an end view of the expandable stent of FIG. 3A in a fully compressed configuration;

FIG. 4A shows a perspective view of a portion of an expandable stent including interlocking eyelets according to a third embodiment;

FIG. 4B shows a flattened plan view of the portion of the expandable stent of FIG. 4A;

FIG. 4C shows an end view of the expandable stent of FIG. 4A in a fully compressed configuration;

FIG. 5 shows the expandable stent of FIG. 2A in a radially expanded configuration; and

FIG. 6 is a flow chart showing steps of a method for preparing an expandable stent for delivery into a body vessel.

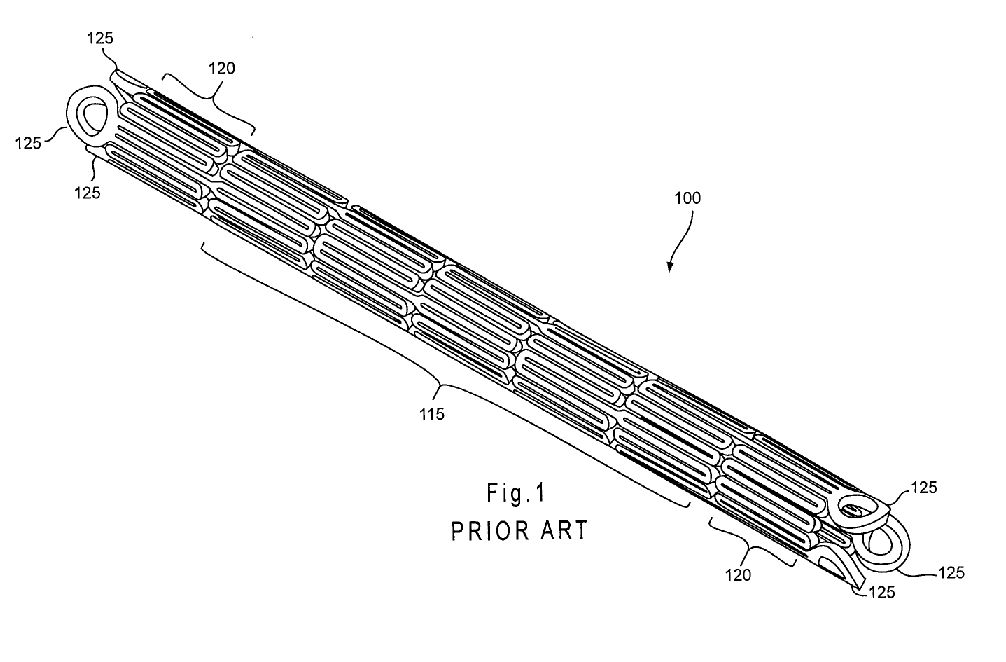

### DETAILED DESCRIPTION

Described herein are expandable stents having end members that interlock when the stent is in a reduced diameter configuration. Such an interlocking configuration may provide advantages when the stent is under an axial load, such as during transfer of the stent out of a compression apparatus or during deployment of the stent. In particular, axial forces applied to the end members may be more uniformly distributed about the circumference of the stent, thus reducing the likelihood of damage during transfer or deployment.

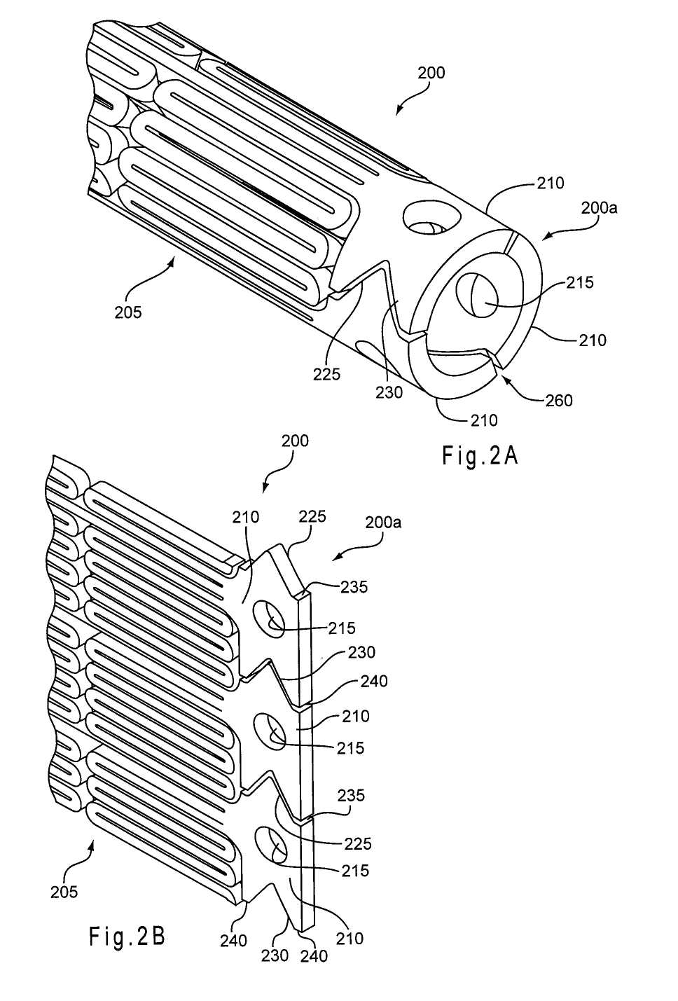

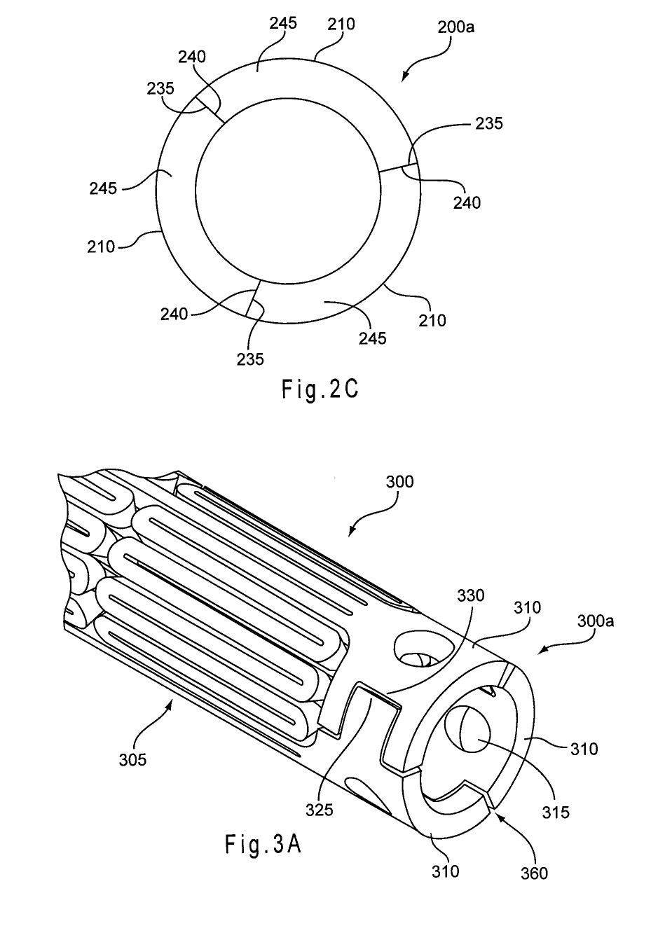

In each of FIGS. 2A, 3A, and 4A, a perspective view of a portion of an expandable stent having interlocking end members is shown. The end members shown are eyelets that include openings for containing radiopaque rivets. Such eyelet-rivet structures are referred to as radiopaque markers. The expandable stents are shown in a delivery configuration suitable for passage through a body vessel. The delivery configuration may be attained when the stents are disposed within a tubular delivery system, such as a sheath or catheter. The expandable stents may be self-expanding or balloon-expandable.

Referring first to the embodiment of FIG. 2A, the delivery configuration of the expandable stent 200 comprises a thin-walled tubular framework 205 including three circumferentially adjacent eyelets 210 extending in a longitudinal direction from an end of the framework 205. The circumferentially adjacent eyelets 210 have an interlocking configuration, where a circumferentially directed protrusion 225 of a first eyelet 210 mates with a circumferentially directed recess 230 of a second eyelet 210. As is apparent from FIG. 2A, the mating protrusions 225 and recesses 230 may not be in direct contact in the delivery configuration, but instead may include a small gap 260 between mating surfaces. This is because the delivery configuration of the stent 200 may not correspond to a fully compressed configuration thereof, as will be discussed in more detail below.

Referring to FIG. 2B, which shows a flattened plan view of the portion of the expandable stent 200 shown in FIG. 2A, a first interlocking side 235 of the eyelet 210 includes the protrusion 225 and a second interlocking side 240 includes the recess 230. According to this embodiment, the protrusion 225 has a triangular shape in plan view, and the recess 230 has a mating shape (e.g., a V-shape). The interlocking relationship of the circumferentially adjacent eyelets may be achieved with protrusions and recesses of other interlocking shapes, as will be discussed further below. The protrusion 225 of this embodiment is substantially centered along the first interlocking side 235 of the eyelet 210, and the recess 230 is correspondingly centered along the second interlocking side 240, although an off-center placement of each of the protrusion 225 and the recess 230 is also possible.

Each eyelet 210 includes an opening 215 for containing a radiopaque rivet. In this embodiment of the expandable stent 200, the opening 215 extends entirely through the thickness of the eyelet 210, but it is also contemplated that the opening may extend only partway through the thickness of the eyelet 210. Additionally, while the opening shown in FIG. 2B appears to be substantially circular in shape, the opening is not limited to this configuration. The opening may have other symmetric shapes (e.g., oval, triangular, square or other polygonal shapes), or it may be asymmetric with an arbitrary shape.

Referring to FIG. 2C, which shows an end 200a of the stent 200 in a fully compressed configuration, the circumferentially adjacent eyelets 210 have sides 235, 240 in contact to define a circumferentially continuous edge 245 at the end 200a of the stent 200. The interlocking structure of the eyelets 210 and the circumferentially continuous edge 245 of the stent 200 enhance the robustness of the eyelets 210 when under axial compressive forces, such as during loading of the stent into a transfer tube from a compression apparatus, as will be discussed further below.

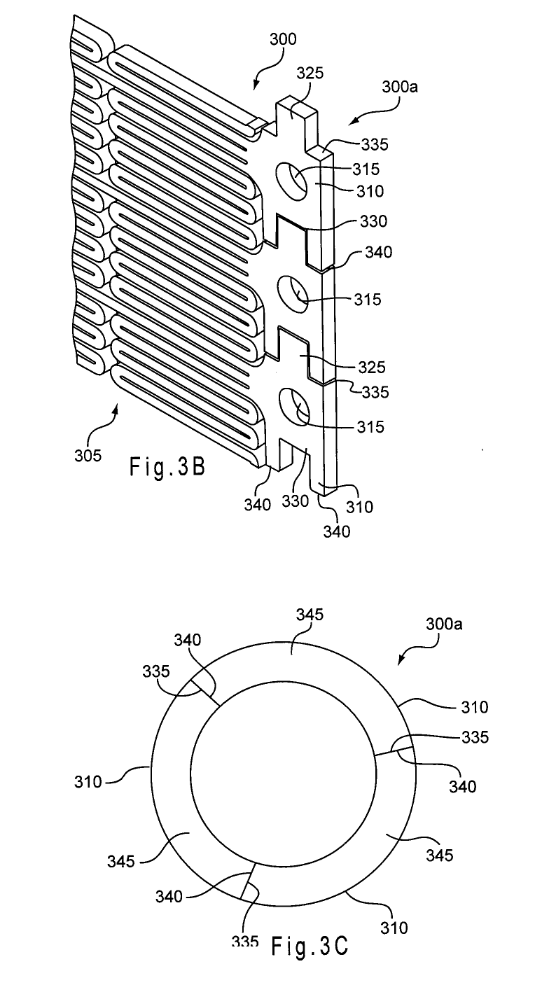

Referring now to the embodiment of FIG. 3A, the delivery configuration of the expandable stent 300 comprises a thin-walled tubular framework 305 including three circumferentially adjacent eyelets 310 extending in a longitudinal direction from an end of the framework 305, as in the previous embodiment. The circumferentially adjacent eyelets 310 have an interlocking configuration, where a circumferentially directed protrusion 325 of a first eyelet 310 mates with a circumferentially directed recess 330 of a second eyelet 310. The mating protrusions 325 and recesses 330 may not be in direct contact in the delivery configuration, but instead may include a small gap 360 between mating surfaces. This is because the delivery configuration of the stent 300 may not correspond to a fully compressed configuration thereof.

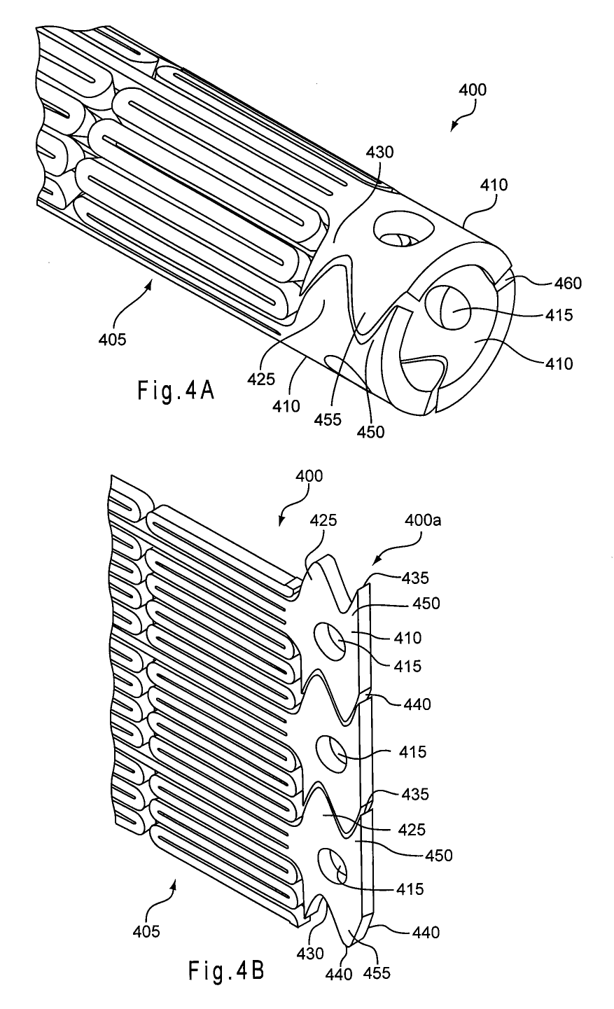

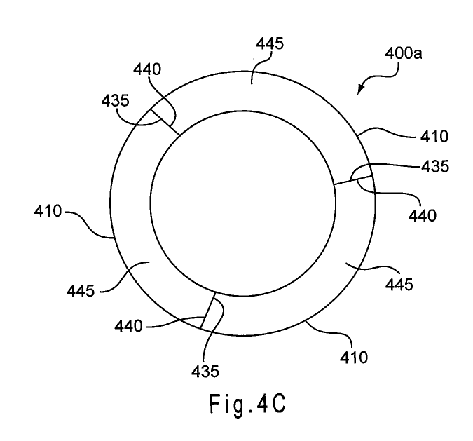

Referring to FIG. 3B, which shows a flattened plan view of the portion of the expandable stent 300 shown in FIG. 3A, a first interlocking side 335 of the eyelet 310 includes the protrusion 325 and a second interlocking side 340 includes the recess 330. According to this embodiment, the protrusion 325 has a rectangular shape in plan view, and the recess 330 has a mating shape (e.g., an angled U-shape). The protrusion 325 is substantially centered along the first interlocking side 335 of the eyelet, and the recess 330 is correspondingly centered along the second interlocking side 340. Alternatively, the protrusion 325 and the recess 335 may not be centered along the first and second interlocking sides 335, 340, respectively, as will be discussed further below in regard to the expandable stent of FIGS. 4A-4B.

Each eyelet 310 includes an opening 315 for containing a radiopaque rivet. As in the previous embodiment, the opening 315 extends entirely through the thickness of the eyelet 310; however, it is also contemplated that the opening may extend only partway through the thickness of the eyelet 310. Additionally, while the opening shown in FIG. 3B is substantially circular in shape, the opening is not limited to this configuration. The opening may have other symmetric shapes (e.g., oval, triangular, square or other polygonal shapes), or it may be asymmetric with an arbitrary shape.

Referring now to FIG. 3C, which shows an end 300a of the stent 300 in a fully compressed configuration, the circumferentially adjacent eyelets 310 of the expandable stent 300 have sides in contact to define a circumferentially continuous edge 345 at the end 300a of the stent 300, as in the previous embodiment. The interlocking structure of the eyelets 310 and the circumferentially continuous edge 345 of the stent 300 enhance the robustness of the eyelets 310 when under axial compressive forces, such as during loading of the stent into a transfer tube from a compression apparatus.

FIGS. 4A and 4B show an alternative embodiment of the expandable stent 400 in a delivery configuration. As in the previous two embodiments, the delivery configuration of the expandable stent 400 comprises a thin-walled tubular framework 405 including three circumferentially adjacent eyelets 410 extending in a longitudinal direction from an end of the framework 405. The circumferentially adjacent eyelets 410 have an interlocking configuration, where a circumferentially directed protrusion 425 of a first eyelet 410 mates with a circumferentially directed recess 430 of a second eyelet 410. The opening 415 of the eyelet 410 for containing a radiopaque rivet is as described in the previous embodiments.

FIG. 4B shows a flattened plan view of the portion of the expandable stent 400 shown in FIG. 4A. According to this embodiment, the protrusion 425 has an arched or curved shape in plan view, and the recess 430 has a curved mating shape (e.g., a U-shape), in contrast to the angular shapes of the protrusions and recesses of FIGS. 2A-3B.

Referring to both of FIGS. 4A and 4B, a first interlocking side 435 of the eyelet 410 includes not only the protrusion 425, but also a circumferentially directed secondary recess 450. Similarly, a second interlocking side 440 of the eyelet 410 includes not only the recess 430, but also a circumferentially directed secondary protrusion 455. Accordingly, a sinusoidal type of pattern is formed along each of the first and second interlocking sides 435, 440 by the presence of both a protrusion and a recess. Just as the protrusion 425 of the first interlocking side mates with the recess 430 of the second interlocking side, the secondary protrusion 455 mates with the secondary recess 450 when the expandable stent 400 is in a delivery or a compressed configuration.

FIG. 4C shows an end 400a of the stent 400 in a fully compressed configuration. As in the previous embodiments, the circumferentially adjacent eyelets 410 of the expandable stent 400 have sides in contact to define a circumferentially continuous edge 445 at the end 400a of the stent 400. The interlocking structure of the eyelets 410 and the circumferentially continuous edge 445 of the stent 400 enhance the robustness of the eyelets 410 when under axial compressive forces.

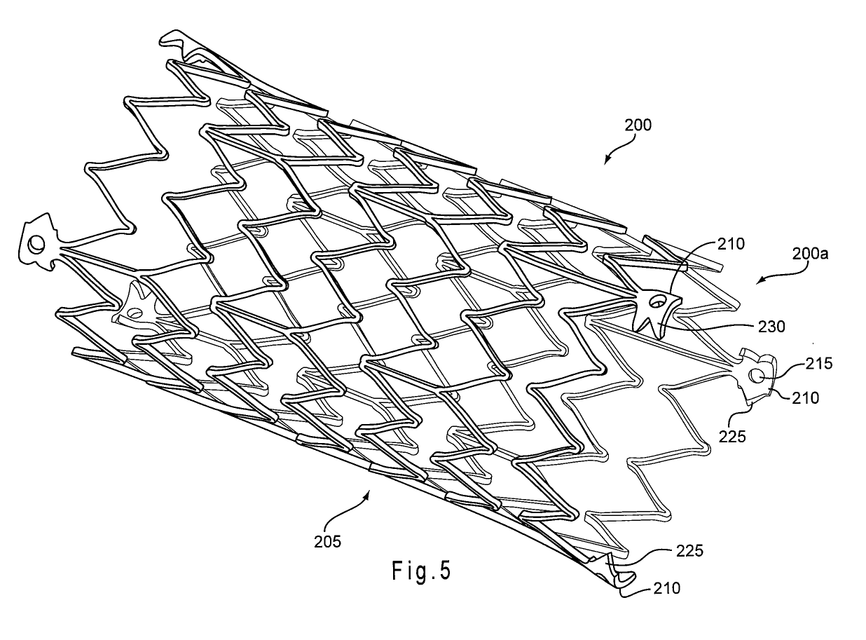

FIG. 5 shows a view of the first embodiment of the expandable stent 200 in an expanded or deployed configuration. As can be seen from the figure, the eyelets 210 are disengaged from the interlocking configuration when the stent 200 is radially expanded from a compressed state.

The expandable stents of the previous embodiments may include circumferentially adjacent end members (e.g., eyelets) extending in a longitudinal direction from one end or from both ends of the tubular framework. Each end of the tubular framework may include two or more end members, three or more end members, four or more end members, or five or more end members. For example, the expandable stent may include two, three, four, five, or six end members extending from one end or from each end of the tubular framework.

The expandable stent may be fabricated from a thin-walled tube (or “cannula”) that has been laser cut to selectively remove portions of the tube, leaving a desired pattern of struts and the interlocking end members. The tube may be formed of a biocompatible metal or alloy, such as stainless steel or a Nitinol alloy. The metal or alloy may include at least one element selected from the group consisting of Al, Co, Cr, Cu, Fe, Mn, Mo, Ni, Ti, Zn, and Zr.

The laser cutting may involve loading the thin-walled tube into a laser cutting machine, such as those manufactured by Synova SA of Ecublens, Switzerland, and then cutting a predetermined pattern in the tube using a laser under microprocessor control. Preferably, the laser beam is directed toward the longitudinal axis of the tube. The tube may be translated along and rotated about its longitudinal axis during the laser cutting procedure to form each interlocking end member, including any openings, and the pattern of struts. The size of the beam may be adjusted to affect the fineness of the cut. After cutting, the resulting stent structure may be subjected to secondary processes such as electropolishing and heat setting (the latter specifically in the case of superelastic self-expanding stents) using methods known in the art. The laser cut stent may include a tubular framework of substantially straight segments linked by bent segments that form a zig-zag pattern of interconnected struts about the circumference. Any strut pattern that may be laser cut or otherwise carved out of a thin-walled tube may be suitable for the stent, provided that it provides sufficient radial support to the vessel wall when expanded at the endoluminal treatment site.

Generally, the tube from which the stent is fabricated has an outer diameter that is smaller than an inner diameter of the delivery system into which the stent is to be loaded. In this case, a single cut made with a fine laser beam size may be employed to define the interlocking end members and the strut pattern with no discernible gaps between mating surfaces. For tubes of larger sizes relative to the delivery system, however, it may be advantageous to employ a larger beam size or a series of cuts to form gaps of predetermined sizes between adjacent surfaces when defining the pattern in the stent. Gaps between the mating surfaces of adjacent interlocking end members and neighboring struts in the as-cut stent may allow for further reduction in the diameter of the stent during the compression process. This in turn may facilitate a smooth transfer of the compressed stent into the delivery system. Once in the delivery system, the compressed stent may recoil outward to a delivery configuration of a slightly larger diameter than that of the compressed configuration, depending on the inner diameter of the delivery system. Accordingly, as mentioned previously, the delivery configuration of the stent may include a small gap between mating surfaces that were in direct contact in the compressed configuration.

As discussed above, the end members may be eyelets that include a radiopaque rivet inserted into an opening therein to form a radiopaque marker. Such radiopaque rivets are preferably fabricated from a radiopaque material that is substantially opaque to x-ray radiation and is thus readily visible using an x-ray imaging device, such as a fluoroscope. Preferably, the radiopaque material is also biocompatible. The radiopaque material may include, for example, gold, iridium, niobium, palladium, platinum, silver, tantalum, tungsten, or an alloy thereof.

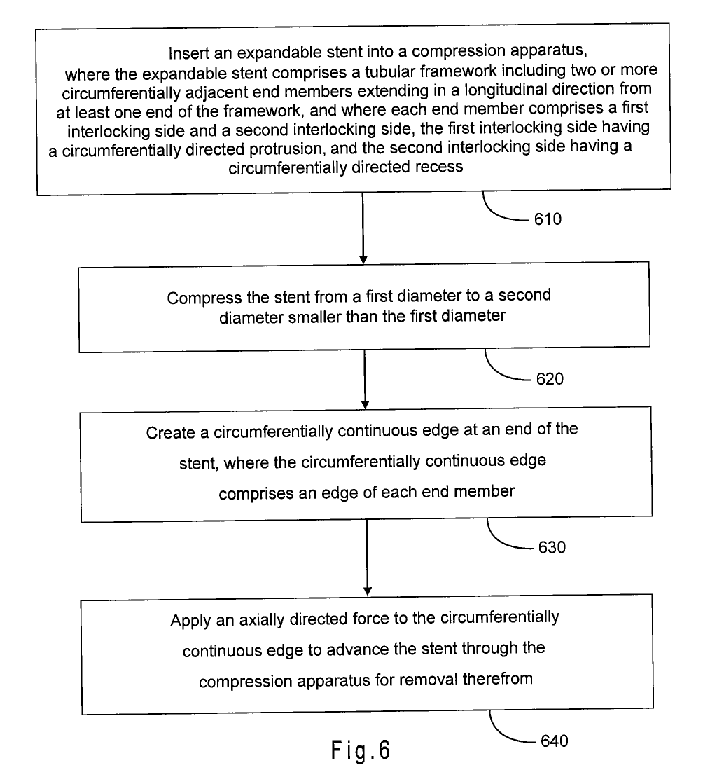

A method of preparing an expandable stent for delivery into a body vessel is described in reference to the flow chart of FIG. 6. The expandable stent comprises a tubular framework including two or more circumferentially adjacent end members extending in a longitudinal direction from a first end of the framework. Each end member comprises a first interlocking side and a second interlocking side, where the first interlocking side has a circumferentially directed protrusion, and the second interlocking side has a circumferentially directed recess. The stent has a first diameter that is larger than a desired delivery diameter.

The stent is inserted 610 into a compression apparatus and is compressed 620 from the first diameter to a second diameter smaller than the first diameter. As the stent is compressed, the end members attain an interlocked configuration in which the protrusion of a first end member mates with the recess of a second end member. Sides of the circumferentially adjacent end members come into contact at the second diameter to create 630 a circumferentially continuous edge at an end of the stent. An axially directed force is applied 640 to the circumferentially continuous edge to advance the stent through the compression apparatus for removal therefrom.

The first diameter from which the expandable stent is compressed may be a radially expanded diameter relative to the diameter of the tube from which the stent was cut, as is typical with self-expanding stents. Alternatively, the first diameter may be substantially the same as the diameter of the tube from which the stent was cut, as is more typical with balloon-expandable stents.

The compression apparatus into which the stent is inserted may be a crimping unit that includes a plurality of contracting members disposed about a cylindrical aperture. The stent may be positioned within the cylindrical aperture and then reduced in diameter as the size of the aperture is decreased by the relative motion of the contracting members. Such crimping units are commercially available from various manufacturers, such as Machine Solutions, Inc. (Flagstaff, Ariz.). Alternatively, the compression apparatus take the form of a stent rolling device which includes a flexible sheet rolled to define a cylindrical opening sized to fit a stent. By applying a tensile force to an end of the sheet with the stent inside the opening, the diameter of the opening may be decreased and the stent may be radially compressed within the sheet. Other compression devices known in the art may also be suitable for the compression.

A push rod or quill may be used to advance the compressed stent out of the compression apparatus and into a tubular delivery system, which may be a sheath, catheter, or transfer tube, in preparation for delivery into a vessel. The push rod contacts the end members as the stent is advanced through the compression apparatus. Due to the substantially continuous circumferential edge formed by the contact between adjacent end members, the axial force may be applied uniformly about the circumference of the stent, and bending or distortion of the end members may be avoided.

Expandable stents having end members that interlock when the stent is in a reduced diameter configuration have been described. Such an interlocking geometry may provide advantages when the stent is under an axial load, such as during transfer of the stent out of a compression apparatus or during deployment of the stent. The interlocking geometry may be particular advantageous when the end members are eyelets with openings for containing radiopaque rivets.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. An expandable stent comprising:
a thin-walled tubular framework including two or more circumferentially adjacent end members extending in a longitudinal direction from an end of the framework,
wherein, in a delivery configuration of the tubular framework, the end members have an interlocking configuration, each end member comprising a first interlocking side and a second interlocking side, the first interlocking side having a circumferentially directed protrusion and the second interlocking side having a circumferentially directed recess, the protrusion of a first end member mating with the recess of a second end member, and
wherein, in an expanded configuration of the tubular framework, the end members are disengaged from the interlocking configuration.

2. The expandable stent of claim 1, wherein each end member further includes an opening for containing a radiopaque rivet, the end member being an eyelet.

3. The expandable stent of claim 2, wherein the opening is a through-thickness opening.

4. The expandable stent of claim 1, wherein the protrusion comprises a curved shape in plan view.

5. The expandable stent of claim 1, wherein the protrusion comprises an angular shape in plan view.

6. The expandable stent of claim 5, wherein the protrusion comprises a rectangular shape in plan view.

7. The expandable stent of claim 5, wherein the protrusion comprises a triangular shape in plan view.

8. The expandable stent of claim 1, wherein the protrusion is centered along the first interlocking side.

9. The expandable stent of claim 1, wherein the first interlocking side further comprises a circumferentially directed secondary recess.

10. The expandable stent of claim 9, wherein the second interlocking side further comprises a circumferentially directed secondary protrusion, the secondary recess of the first interlocking side mating with the secondary protrusion of the second interlocking side.

11. The expandable stent of claim 1, wherein mating surfaces of the protrusion of the first end member and the recess of the second end member include a small gap therebetween, the mating surfaces not being in direct contact in the delivery configuration.

12. The expandable stent of claim 1 wherein, when the stent is fully compressed, the circumferentially adjacent end members have sides in contact at an end of the stent to define a circumferentially continuous edge thereof.

13. The expandable stent of claim 1, comprising at least three circumferentially adjacent end members extending in a longitudinal direction from each end of the tubular framework.

14. The expandable stent of claim 1 being a self-expanding stent.

15. The expandable stent of claim 1, wherein each end member includes an opening for containing a radiopaque rivet therein, each end member being an eyelet,
wherein mating surfaces of the protrusion of the first end member and the recess of the second end member include a small gap therebetween, the mating surfaces not being in direct contact in the delivery configuration, and
wherein, when the stent is fully compressed, the circumferentially adjacent end members have sides in contact at an end of the stent to define a circumferentially continuous edge thereof.

16. The expandable stent of claim 15, comprising at least three circumferentially adjacent end members extending in a longitudinal direction from each end of the tubular framework,
wherein the expandable stent is a self-expanding stent.

17. A method of preparing an expandable stent for delivery into a body vessel, the method comprising:
inserting an expandable stent into a compression apparatus, the expandable stent comprising a thin-walled tubular framework including two or more circumferentially adjacent end members extending in a longitudinal direction from an end of the framework, each end member comprising a first interlocking side and a second interlocking side, the first interlocking side having a circumferentially directed protrusion and the second interlocking side having a circumferentially directed recess;
compressing the stent from a first diameter to a second diameter smaller than the first diameter;
creating a circumferentially continuous edge at an end of the stent, the circumferentially continuous edge comprising an edge of each end member;
applying an axially directed force to the circumferentially continuous edge to advance the stent through the compression apparatus for removal therefrom.

18. The method of claim 17, wherein compressing the stent from a first diameter to a second diameter smaller than the first diameter comprises interlocking the end members by mating the protrusion of a first end member with the recess of a second end member.

19. The method of claim 17, wherein creating the circumferentially continuous edge comprises bringing sides of the end members into contact at the end of the stent.

20. The method of claim 17, wherein the first diameter is a radially expanded diameter of the stent, and
wherein each end member includes an opening for containing a radiopaque rivet therein, each end member being an eyelet.
